(19) United States
(12) Patent Application Publication
(10) Pub. No.: US 2025/0268518 A1
(43) Pub. Date: Aug. 28, 2025

(54) NON-INVASIVE COMPARTMENT SYNDROME DIAGNOSTIC SYSTEM

(71) Applicant: Aspire Medtech Inc., Fountain, CO (US)

(72) Inventors: Tanna Donalson, Golden, CO (US); John Edward Lynch, Williamsburg, VA (US); William Charles Scott, North Palm Beach, FL (US); Yan Sartor Campos, Barrie (CA)

(21) Appl. No.: 19/061,381

(22) Filed: Feb. 24, 2025

Related U.S. Application Data
(60) Provisional application No. 63/557,981, filed on Feb. 26, 2024.

(51) Int. Cl. A61B 5/00; A61B 5/05; A61B 5/145; A61B 8/00; A61B 8/08

(52) U.S. Cl. CPC A61B 5/4538; A61B 5/0095; A61B 5/05; A61B 5/14542; A61B 5/7203; A61B 5/7225; A61B 5/7275; A61B 5/7289; A61B 8/0858; A61B 8/5261

(57) ABSTRACT

A non-invasive compartment syndrome diagnostic system is provided for detecting conditions in a patient indicative of compartment syndrome without the requirement of pulsed phase-locked loop devices or invasive procedures. The non-invasive compartment syndrome diagnostic system may include a radio frequency device, signal conversion device, analytic engine, user terminal, photoacoustic device, multi-modal input signals and imaging stabilization components.

### CROSS-REFERENCE TO RELATED APPLICATION

This application claims the priority from U.S. provisional patent application Ser. No. 63/557,981 filed Feb. 26, 2024. The foregoing application is incorporated in its entirety herein by reference.

### FIELD OF THE INVENTION

The present disclosure relates to a non-invasive compartment syndrome diagnostic system. More particularly, the disclosure relates to detecting conditions in a patient indicative of compartment syndrome without the requirement of pulsed phase-locked loop devices or invasive procedures.

### BACKGROUND

Compartment syndrome is a potentially severe condition that arises when pressure within a muscle compartment increases, thereby jeopardizing tissue perfusion and function. The early detection and subsequent intervention of compartment syndrome are essential to prevent irreversible damage. In recent times, the pulse phase-locked loop (PPLL) system has emerged as a technique to detect changes in the pulsatile components of tissue pressure, signifying compartmental pressure shifts. PPLL works by tracking the phase of a pulsating signal and subsequently controlling another system to keep it synchronized with the pulsating signal's phase. In the context of compartment syndrome, PPLL monitors the phase alterations in the arterial pulse wave within a compartment, providing real-time feedback on internal pressures.

However, as with many emerging techniques, the use of PPLL in detecting compartment syndrome is not without its deficiencies. First and foremost, the sensitivity and specificity of PPLL in detecting pressure changes might not be adequate for all clinical scenarios. As with many diagnostic tools, false positives or negatives could lead to unnecessary interventions or missed opportunities for treatment, respectively. Additionally, while PPLL provides information on pulsatile components of compartment pressures, it may not capture the complete picture, particularly in conditions where non-pulsatile factors significantly impact compartment pressures.

Moreover, the technical complexities associated with PPLL might present challenges in its widespread adoption. Not all medical professionals may be familiar or comfortable with the intricacies of PPLL, requiring extensive training and standardization across medical institutions. Furthermore, factors such as patient movement, external vibrations, or other confounders might introduce noise into the PPLL readings, decreasing its reliability in dynamic, real-world settings. It is thus essential for further research and development to address these challenges and assess the true clinical value of PPLL in the early detection and management of compartment syndrome.

Therefore, a need exists to solve the deficiencies present in the prior art. What is needed is a system to detect indicators of compartment syndrome without requiring use of pulse phase-locked loop devices or invasive procedures. What is needed is a system to assist physicians with diagnosing compartment syndrome. What is needed is a system using ultrasonic frequencies to assist with the prediction of compartment syndrome within a patient. What is needed is a noninvasive system for assisting with the detection of compartment syndrome in a patient. What is needed is a system for using a multi-modal approach to diagnose risk relating to compartment syndrome and other adverse medical conditions.

### SUMMARY

An aspect of the disclosure advantageously provides a system to detect indicators of compartment syndrome without requiring use of pulse phase-locked loop devices or invasive procedures. An aspect of the disclosure advantageously provides a system to assist physicians with diagnosing compartment syndrome. An aspect of the disclosure advantageously provides a system using ultrasonic frequencies to assist with the prediction of compartment syndrome within a patient. An aspect of the disclosure advantageously provides a noninvasive system for assisting with the detection of compartment syndrome in a patient. An aspect of the disclosure advantageously provides a system for using a multi-modal approach to diagnose risk relating to compartment syndrome and other adverse medical conditions.

Accordingly, the disclosure may feature a non-invasive adverse medical condition diagnostic system including a radio frequency (RF) device and analytic engine. The RF device may include an electro-acoustic transducer to emit and detect radio signals being at least partially within an ultrasound frequency range to detect responsive displacements of the tissue resulting from induced displacements over a sampling duration. The analytic engine may interpret digital signal data indicative of the radio signals to derive diagnostic information from at least a displacement relationship between the responsive displacements of the tissue reacting to the induced displacements throughout at least part of the sampling duration. A risk of an adverse medical condition may be indicated via interpretation of at least the displacement relationship.

In another aspect, the tissue may include artery tissue, compartment tissue, and fascia tissue. The adverse medical condition may include compartment syndrome. Risk may be categorized into groups including: a low risk indicated by detecting high displacement amplitude in the artery tissue, low displacement amplitude in the compartment tissue, and low displacement amplitude in the fascia tissue; a medium risk indicated by detecting high displacement amplitude in the artery tissue, high displacement amplitude in the compartment tissue, and high displacement amplitude in the fascia tissue; and a high risk indicated by detecting low displacement amplitude in the artery tissue, low displacement amplitude in the compartment tissue, and low displacement amplitude in the fascia tissue.

In another aspect, a user terminal may be included to present at least part of the diagnostic information derived by the analytic engine to an operator to indicate a likelihood of development of the adverse medical condition.

In another aspect, the user terminal may include a tilt sensor to assist with orienting the electro-acoustic transducer to optimize efficacy by which the radio signals apply the induced displacements to the tissue and detect the responsive displacements of the tissue.

In another aspect, a signal conversion device may be provided to adapt the radio signals detected by the RF device from an analog electrical signal to the digital signal data. The signal conversion device may include filters to reduce unwanted interference comprising signal noise from the radio signals that are detected by the RF device to enhance the efficacy by which the digital signal data is interpreted by the analytic engine.

In another aspect, the filters may use digital signal processing to at least partially filter the digital signal data.

In another aspect, the radio signals may be gated with an electrocardiogram (ECG) to substantially correlate the induced displacement and responsive displacement with electrical cardiac activity respective to a point in a cardiac cycle. The analytic engine may temporarily align and interpret a relationship between the point in the cardiac cycle and the digital signal data while deriving the diagnostic data.

In another aspect, the analytic engine may interpret the responsive displacements of the tissue during systole and diastole of the cardiac cycle as indicated by the ECG.

In another aspect, the radio signals may be emitted and received by the RF device using non-uniform sampling. The radio signals may be substantially correlated with the point in the cardiac cycle via gating with the ECG.

In another aspect, a photoacoustic device may be provided to detect blood oxygenation data to supplement the radio signals of the RF device. The analytic engine may interpret a physiological state of the tissue indicated by the blood oxygenation data for the diagnostic information.

In another aspect, the electro-acoustic transducer may further include a light emitting device communicably connected with the photoacoustic device to detect the blood oxygenation data synchronously with detection of the radio signals by the RF device.

In another aspect, the analytic engine may derive the diagnostic data using multi-modal input signals comprising at least two selected from the group consisting of: ultrasound signals, acoustic radiation force impulse (ARFI) signals, photoacoustics, and/or ECG gating signals.

In another aspect, registration tracing may be applied to identify a landmark of the radio signals and at least partially align the radio signals over time by matching the landmark to substantially stabilize the digital data signal used by the analytic engine to derive the diagnostic information.

In another aspect, the radio signal may be detected by the RF device using B-mode to perform full image sampling of the tissue included within a sampling area.

According to an additional embodiment, the disclosure may feature a non-invasive compartment syndrome diagnostic system including an RF device, a signal conversion device, and an analytic engine. The RF device may include a transducer to emit and detect radio signals to apply induced displacements in tissue and detect responsive displacements of the tissue over a sampling duration. The signal conversion device may adapt the radio signals detected by the RF device from an analog electrical signal to digital signal data, the signal conversion device comprising filters to reduce unwanted interference comprising signal noise from the radio signals that are detected by the RF device. The analytic engine may interpret the digital signal data to derive diagnostic information from at least a displacement relationship between the responsive displacements of the tissue reacting to the induced displacements throughout at least part of the sampling duration. The tissue may include artery tissue, compartment tissue, and fascia tissue. The risk of compartment syndrome may be indicated via interpretation of at least the displacement relationship, which may be categorized into risk groups including a low risk indicated by detecting high displacement amplitude in the artery tissue, low displacement amplitude in the compartment tissue, and low displacement amplitude in the fascia tissue; a medium risk indicated by detecting high displacement amplitude in the artery tissue, high displacement amplitude in the compartment tissue, and high displacement amplitude in the fascia tissue; and a high risk indicated by detecting low displacement amplitude in the artery tissue, low displacement amplitude in the compartment tissue, and low displacement amplitude in the fascia tissue.

In another aspect, a user terminal may be provided to present at least part of the diagnostic information derived by the analytic engine to an operator to indicate a likelihood of development of compartment syndrome. A tilt sensor may be included by the user terminal to assist with orienting the transducer to optimize efficacy by which the radio signals apply the induced displacements to the tissue and detect the responsive displacements of the tissue.

In another aspect, the radio signals may be gated with an electrocardiogram (ECG) to substantially correlate the induced displacement and responsive displacement with electrical cardiac activity respective to a point in a cardiac cycle. The analytic engine may temporarily align and interpret a relationship between the point in the cardiac cycle and the digital signal data while deriving the diagnostic data. The radio signals may be emitted and received by the RF device using non-uniform sampling. The radio signals may be substantially correlated with the point in the cardiac cycle via gating with the ECG.

In another aspect, registration tracing may be applied to identify a landmark of the radio signals and at least partially align the radio signals over time by matching the landmark and substantially stabilizing the digital data signal used by the analytic engine to derive the diagnostic information.

According to an additional embodiment, the disclosure may feature a non-invasive adverse medical condition diagnostic system including an RF device and an analytic engine. The radio frequency (RF) device may include an ultrasonic transducer to emit and detect radio signals being at least partially within an ultrasound frequency range to apply induced displacements in tissue and detect responsive displacements of the tissue over a sampling duration. The analytic engine may interpret digital signal data indicative of the radio signals to derive diagnostic information from at least a displacement relationship between the responsive displacements of the tissue reacting to the induced displacements throughout at least part of the sampling duration. The radio signals may be gated with an electrocardiogram (ECG) to substantially correlate the induced displacement and responsive displacement with electrical cardiac activity respective to a point in a cardiac cycle. The analytic engine may temporarily align and interpret a relationship between the point in the cardiac cycle and the digital signal data while deriving the diagnostic data. A risk of an adverse medical condition may be indicated via interpretation of at least the displacement relationship.

In another aspect, the radio signal may be detected by the RF device using B-mode to perform full image sampling of the tissue included within a sampling area.

Terms and expressions used throughout this disclosure are to be interpreted broadly. Terms are intended to be understood respective to the definitions provided by this specification. Technical dictionaries and common meanings understood within the applicable art are intended to supplement these definitions. In instances where no suitable definition can be determined from the specification or technical dictionaries, such terms should be understood according to their plain and common meaning. However, any definitions provided by the specification will govern above all other sources.

Various objects, features, aspects, and advantages described by this disclosure will become more apparent from the following detailed description, along with the accompanying drawings in which like numerals represent like components.

### BRIEF DESCRIPTION OF THE DRAWINGS

FIG. 1 is a block diagram view of an illustrative system for non-invasive diagnosis of adverse medical conditions such as compartment syndrome, according to an embodiment of this disclosure.

FIG. 2 is a graph view of a two-dimensional view of an indication for low risk to medium risk of compartment syndrome using a linear RF signal, according to an embodiment of this disclosure.

FIG. 3 is a graph view of a three-dimensional view of an indication for low risk to medium risk of compartment syndrome using a linear RF signal correlating with FIG. 2, according to an embodiment of this disclosure.

FIG. 4 is a graph view of a two-dimensional view of an indication for low risk of compartment syndrome using a linear RF signal, according to an embodiment of this disclosure.

FIG. 5 is a graph view of a three-dimensional view of an indication for low risk of compartment syndrome using a linear RF signal correlating with FIG. 4, according to an embodiment of this disclosure.

FIG. 6 is a graph view of a two-dimensional view of an indication for medium risk of compartment syndrome using a linear RF signal, according to an embodiment of this disclosure.

FIG. 7 is a graph view of a three-dimensional view of an indication for medium risk of compartment syndrome using a linear RF signal correlating with FIG. 6, according to an embodiment of this disclosure.

FIG. 8 is a block diagram view of an illustrative interface provided by a user terminal, according to an embodiment of this disclosure.

FIG. 9 is a diagram view of an illustrative computerized device upon which some aspects of a system enabled by this disclosure may be operated, according to an embodiment of this disclosure.

### DETAILED DESCRIPTION

The following disclosure is provided to describe various embodiments of a non-invasive compartment syndrome diagnostic system. Skilled artisans will appreciate additional embodiments and uses of the present invention that extend beyond the examples of this disclosure. Terms included by any claim are to be interpreted as defined within this disclosure. Singular forms should be read to contemplate and disclose plural alternatives. Similarly, plural forms should be read to contemplate and disclose singular alternatives. Conjunctions should be read as inclusive except where stated otherwise.

Expressions such as “at least one of A, B, and C” should be read to permit any of A, B, or C singularly or in combination with the remaining elements. Additionally, such groups may include multiple instances of one or more elements in that group, which may be included with other elements of the group. All numbers, measurements, and values are given as approximations unless expressly stated otherwise.

For the purpose of clearly describing the components and features discussed throughout this disclosure, some frequently used terms will now be defined, without limitation. The term compartment syndrome, as it is used throughout this disclosure, is defined as a dangerous medical condition in which increased pressure within a muscle compartment compromises blood flow and can lead to muscle and nerve damage.

The term tissue, as it is used throughout this disclosure, is defined as biological material, which includes tissue within a muscle compartment, fascia, fibers, nerves blood vessels, connective tissue, and other biological materials that would be apparent to a person of skill in the art. The term artery, as it is used throughout this disclosure, is defined as a blood vessel that carries oxygenated blood away from the heart for delivery to a tissue. The term compartment, as it is used throughout this disclosure, is defined as a section of a body containing muscles, blood vessels, and nerves, bound by fascia or bone, that can be subject to increased internal pressures in certain conditions. The term facia, as it is used throughout this disclosure, is defined as a band or sheet of connective tissue, primarily made up of collagen, that surrounds muscles, groups of muscles, blood vessels, and nerves, binding them together and defining compartments in the body.

The term radio signals, as it is used throughout this disclosure, is defined as information provided by a wave of electromagnetic radiation, which includes acoustic waves and radio signals within the ultrasound frequencies. The term digital signal processing, as it is used throughout this disclosure, is defined as mathematical manipulation of digital signals to analyze, modify, or transform information using algorithms. The term photoacoustics, as it is used throughout this disclosure, is defined as generation of acoustic waves as a result of the absorption of modulated or pulsed light by a material, often used in biomedical imaging and material characterization. The term electrocardiogram (ECG), as it is used throughout this disclosure, is defined as a technique to measure electrical activity of a heart. The term cardiac cycle, as it is used throughout this disclosure, is defined as a sequence of events that occur during a heart beat, including systole and diastole phases.

Various aspects of the present disclosure will now be described in detail, without limitation. In the following disclosure, a non-invasive compartment syndrome diagnostic system will be discussed. Those of skill in the art will appreciate alternative labeling of the non-invasive compartment syndrome diagnostic system as a medical condition diagnosis assistance system, non-invasive compartment syndrome detection assistance system, compartment inspection system, the invention, or other similar names. Similarly, those of skill in the art will appreciate alternative labeling of the non-invasive compartment syndrome diagnostic system as a compartment syndrome detection assistance method, method of using radio frequency to assist with detection of compartment syndrome, medical condition detection method, method, operation, the invention, or other similar names. Skilled readers should not view the inclusion of any alternative labels as limiting in any way.

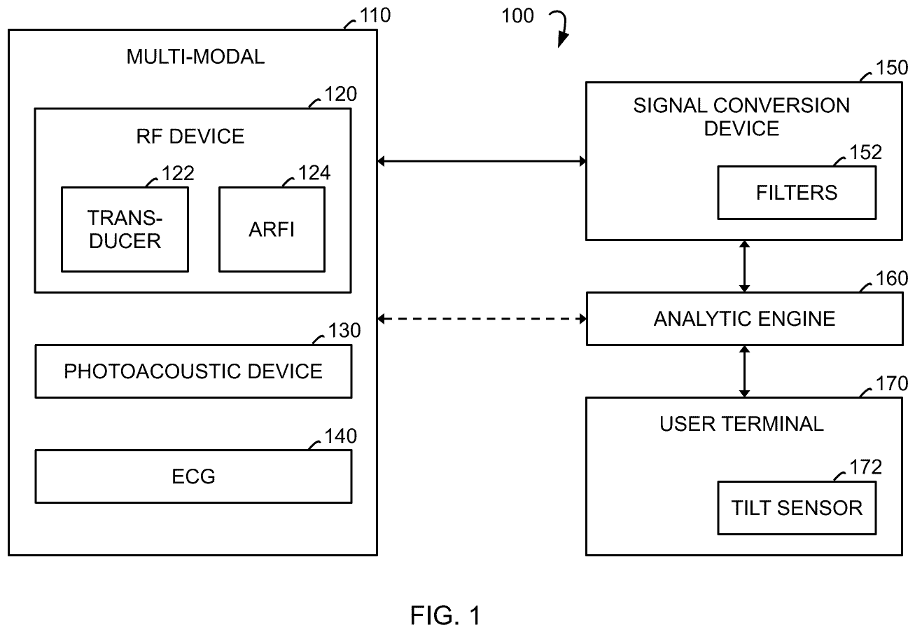

Referring now to FIGS. 1-9, the non-invasive compartment syndrome diagnostic system will now be discussed in more detail. The non-invasive compartment syndrome diagnostic system 100 may include multi-modal input signals 110 such as a radio frequency device 120 and photoacoustic device 130, signal conversion device 150, analytic engine 160, user terminal 170, imaging stabilization components, and additional components that will be discussed in greater detail below. The non-invasive compartment syndrome diagnostic system may operate one or more of these components interactively with other components for detecting conditions in a patient indicative of compartment syndrome without the requirement of pulsed phase-locked loop devices or invasive procedures.

The disclosure generally relates to a novel device and system to assist with detecting compartment syndrome and/or other adverse medical conditions in a patient. In some embodiments, the system may assist with detecting biomarkers relating to a patient, for example, pulsatility. The invention focuses on flexible, digital analysis of activity within a compartment. In some embodiments, the analysis may use signal displacements and/or wave velocity to first determine tissue velocity, which then over time may show the tissue displacements. A system enabled by this disclosure may improve on prior art techniques that disadvantageously require physical electronic filters to transform a signal using PPLL by performing equivalent or enhanced calculations on flexible computing platforms.

A system enabled by this disclosure may use other aspects of the tissue and/or vasculature for detecting and staging risk relating to compartment syndrome. In various embodiments, the following may be used by themselves or in combination for detecting and staging compartment syndrome: total volume flow through the vasculature, an analysis of the pulsatility of the blood moving through the vasculature, arterial pulsatility, fascial pulsatility, and monitoring of other elements, without limitation. In some embodiments, the natural operation of the cardiovascular system may create induced displacements as the heart operates within the cardiac cycle. In other embodiments, the system may apply induced displacements in the tissue, for example, via a transceiver of the RF device 120. Various ways this may be done include using Acoustic Radiation Force Imaging (ARFI) 124 for direct measurements of stiffness, measure viscoelasticity, or taking relative measurements of stiffness. Those skilled in the art will appreciate the various other ways the system may induce displacements in the tissue. In the same or different embodiment, the blood pulsatility may be used as a source of the induced displacements in place of an ARFI push for similar displacement analysis.

The radio frequency device will now be discussed in greater detail. FIG. 1 highlights examples of the radio frequency (RF) device, which may also be shown in other figures. As will be appreciated by those of skill in the art, a radio frequency (RF) device, may be provided to emit and/or detect a radio signal. The radio signal associated with the RF device 120 may be an acoustic signal, such as may be related to ultrasound. Signals may operate to perform spectral doppler and/or shear wave imaging. Signals associated with the RF device 120 may use different display systems and processing to leverage movement-mode (M-mode) and brightness mode (B-mode) to provide insight into characteristics of signal displacements in compartments.

In one embodiment, the RF device 120 may emit ultrasonic radio signals. Alternative embodiments can exist, for example, including a distinct RF emitter or including photoacoustic devices 130, without limitation. In some embodiments, the radio signals may be used to sample a region of interest being linear, conical, included by a sampling area, or otherwise configured as will be appreciated by a person of skill in the art. In embodiments that include full image sampling, radio signals may be acquired from an entire region of interest (ROI) to create a comprehensive image of the underlying tissues by systematically scanning the ultrasound beam across the ROI and recording the echoes reflected from different tissue interfaces.

A system enabled by this disclosure may operate by sending a series of emitted radio signals to detect displacement/movement from one or more locations inside and/or on the surface of a compartment due to the change in arterial pressure over one or more segments of one or more heartbeats. Signals can be sent and detected from an arterial location as a reference for tissue displacements relating to the compartment. In an alternative embodiment, the emitted radio signals may indicate landmarks such as arterial, venous, fascia walls may be chosen from points in or around the compartment. One or more locations or regions may be interrogated within the compartment to monitor the distribution of displacement amplitudes throughout the compartment.

In some embodiments, if a transducer 122 is used, such as an ultrasound and/or electro-acoustic transducer, it may be positioned approximately perpendicular to the surface of the location being observed with two dimensions of tilt. For example, the transducer 122 may be positioned ranging between about perpendicular to about 60-degrees from the ultrasound beam. Optionally, the transducer angle can be indicated using a tilt gauge included by the user terminal 170. In some embodiments, an elevational indicator may be used to obtain a maximum angle, which may advantageously assist with accounting for user error by reducing total displacement when the angle is off. Optionally, a rock indicator may alternatively be used to obtain the maximum signal from the fascia. This may be automated through multi-line acquisition methods, such as a gate angle, adjusted by the user similar to pulse width (PW) doppler for compensation. A vector doppler approach can compensate with the use of specific transmitted waveforms to generate lateral spatial sinusoids. Alternatively, 2D cross correlation can be used for compensation, for example, via 2D speckle tracking. In another embodiment, plane wave imaging may be used to emit various angles into the tissue, which may advantageously allow for automatic angle corrections.

The RF device 120 may use M-mode signals to detect movement of the artery and fascia of a compartment. In this context, M-Mode may describe a method of user interface interaction. Additional information can be received by sending B-mode signals to supplement and optimize the M-mode signals. In embodiments using full image sampling, B-mode signals may be used to survey sampling areas. However, these two modes can function independently in some embodiments. Using B-mode signals may assist with detecting general movement and may enhance information for displacement of the fascia. In some applications, the M-mode and/or B-mode signals may be sent and detected at no less than 2× the range of the highest frequency of the heart rate, which is during the diastole phase of the heartbeat. Optionally, the ultrasound may be gated with an ECG-type device to reduce the total number of samples needed for analysis, which may further be used for filtering the data and/or other processing. Embodiments including gating with ECG 140 will be discussed in greater detail below.

Various reflected radio signals, for example a first reflected radio signal and second reflected radio signal, may then be compared over time to derive a velocity value. The radio signals may be correlated, for example, using autocorrelation, cross-correlation, principal component analysis, and/or other analytical processes. Autocorrelation (AC) may allow improved accuracy for measuring displacements down to ˜0.5 microns (˜0.0005 mm). In various embodiments, however, the AC may allow to accurately measure displacements lower than 0.5 microns.

Filters 152, such as digitally-applied filters, can be used with the information received from the reflected radio signals to focus on the information desired, eliminate user interference, and reduce background noise. Peaks can be identified in the filter 152 to indicate an event, for example, the pulsing of an artery. The event data may be compared to an observation of the fascia for a compartment to determine a discontinuity between the displacement of the facia, the displacement ratio between the fascia and the artery wall, or other number of points in between.

In some embodiments, filtering may be used to reduce system noise and other interferences, which may advantageously increase the resolution of displacements measurable by a system enabled by this disclosure. However, other methods may be used in place, including normalized complex cross-correlation/covariance, principal component analysis, and/or frequency domain phase rotation, without limitation. Collecting a variety of samples over time may advantageously be used through various tests. In various embodiments, the samples may be used in a pulsed wave doppler processing, which may convert time domain samples into a frequency domain at one or more locations. In the same or other embodiments, these samples may further track the changes over time in the frequency domain, without limitation.

The system may compare diagnostic information resulting from the radio signals, including calculating the displacement between samples to obtain displacement per unit of time and a first derivative of position (velocity/slope). The slope of the lines may be preferred to overcome the probe/tissue moving elevationally or laterally, as these can corrupt the correlation between the lines that are too far apart in time. Lines may be correlated from one to the next, which yields the first derivative of the displacement to be integrated over time to get back to the actual displacement magnitude.

Displacement filtering may be performed to filter 152 the calculate displacement value and can be accomplished via various filtering techniques, for example, bandpass Finite Impulse Response (FIR), Infinite Impulse Response (IIR), regression, Principal Component Analysis (PCA), and/or Singular Value Decomposition (SVD). A high pass filter may be applied to remove most of the patient/operator motion. A low pass filter may be applied to remove higher frequency noise. Multiple heartbeat cycles could be segmented in the displacement data to average out noise. Correlations between combinations of additional lines may be used to improve the signal.

In some embodiments, the RF device 120 may include an electro-acoustic transducer 122 to emit and detect radio signals being at least partially within an ultrasound frequency range to apply induced displacements in tissue and detect responsive displacements of the tissue over a sampling duration. The radio signal may be detected by the RF device 120 using B-mode to perform full image sampling of the tissue included within a sampling area. For example, a system enabled by this disclosure may utilize an RF device 120 equipped with an electro-acoustic transducer 122 to emit and detect radio signals, which may be primarily within the ultrasound frequency range, to induce displacements in the tissue and measure the responsive displacements over a specific sampling duration. In one embodiment of the system, the RF device 120 employs B-mode imaging to perform full image sampling of the tissue within a defined sampling area.

Using an electro-acoustic transducer 122 may emit, receive, and convert between electrical energy and acoustic energy. In transmit mode, the transducer 122 may convert electrical pulses into ultrasound waves that propagate through tissue. In receive mode, the transducer 122 may detect the reflected ultrasound waves and converts them back into electrical signals. Radio signals may be emitted and detected by the RF device 120 primarily within the ultrasound frequency range, which advantageously penetrate tissues and interact with their structures, providing valuable information about their properties and dynamics. In some embodiments, the RF device 120 may induce displacements in the tissue by emitting short bursts of ultrasound waves, for example, using ARFI 124. These waves exert a force on the tissue, causing it to deform and move. The transducer 122 then detects the responsive displacements of the tissue, measuring how it moves in response to the induced force.

The RF device 120 may measure the tissue displacements over a sampling duration, which is the time interval during which the transducer 122 actively acquires data, which may be chosen to capture the relevant tissue dynamics such as the pulsatile motion caused by the heartbeat and/or the response to the ARFI push.

In one embodiment of the system, the RF device 120 may use B-mode imaging to acquire full image data of the tissue within a defined sampling area. B-mode imaging may create a two-dimensional grayscale image of the tissues, providing information about their structure and morphology. This information can be used to identify anatomical landmarks, assess tissue health, and guide the placement of the transducer 122 for more targeted measurements. Use of B-mode imaging may advantageously provide a comprehensive view of the tissues within the sampling area, allowing for a more complete assessment of tissue health. Use of B-mode for full image sampling may additionally enable identification of anatomical landmarks, which can be used to guide the placement of the transducer 122 for more targeted measurements. Furthermore, B-mode imaging may assist with assessing tissue morphology and identifying abnormalities that may be indicative of compartment syndrome and/or other adverse medical conditions. The B-mode imaging data may also be integrated with other modalities, such as Doppler and photoacoustics, to assess a muscle compartment.

The signal conversion device will now be discussed in greater detail. FIG. 1 highlights examples of the signal conversion device 150, which may also be shown in other figures. The signal conversion device 150 may adapt the radio signals detected by the RF device 120 from an analog electrical signal to the digital signal data. Filters 152, for example using digital signal processing, may be included to reduce unwanted interference comprising signal noise from the radio signals that are detected by the RF device 120 to enhance the efficacy by which the digital signal data is interpreted by the analytic engine 160.

The signal conversion device 150 may convert the analog electrical signals generated by the RF device 120 into digital signals that can be processed and analyzed by the analytic engine 160. The analog signals produced by the RF device 120 represent the raw ultrasound echoes reflected from the tissues within the muscle compartment. These signals may be continuous in nature, varying in amplitude and frequency over time. To be digitally processed by the analytic engine 160, analog signals may be converted by the signal conversion device 150 and/or otherwise into a discrete, numerical format.

The signal conversion device 150 may perform signal conversion operations via analog-to-digital conversion, which may sample analog signals at regular intervals and quantize the sampled values into discrete digital levels. The sampling rate and the number of quantization levels determine the accuracy and resolution of the digital representation of the analog signal. The digital signals produced by the signal conversion device 150 may then be communicated to the analytic engine 160 for further processing and analysis. The signal conversion device 150 may also perform other signal conditioning tasks, such as filtering and amplification. Filtering can be used to remove unwanted noise and artifacts from the analog signal before conversion, improving the quality of the digital data. Amplification can be used to boost the strength of weak signals, ensuring that they are adequately represented in the digital domain.

The analytic engine 160 will now be discussed in greater detail. FIG. 1 highlight examples of the analytic engine 160, which may also be shown in other figures. The analytic engine 160 may interpret digital signal data indicative of the radio signals, which may be received from the signal conversion device 150, to derive diagnostic information from at least a displacement relationship between the responsive displacements of the tissue reacting to the induced displacements throughout at least part of the sampling duration.

The analytic engine 160 may be operable from memory of a computerized device to analyze the output signal and calculate a displacement signal within the compartment and/or a local fascia to compare with reference values. The user terminal 170 may be provided for guiding the operator and displaying the displacement amplitude, waveform, frequency content, area, and/or volume changes over time from one or more heartbeats from the one or more displacement signals. The displacement amplitude of the blood vessel, tissue, and/or local fascia may be correlated with reference values. The distribution profile of the displacement through the compartment may also be correlated with reference values. The displacement signals through the compartment may be tracked over time to indicate stages of compartment expansion and/or increasing internal pressure. In some embodiments, the analytic engine 160 may calculate displacement and apply filters 152 to reduce noise and/or perform autocorrelation to accurately measure displacement.

The analytic engine 160 may receive input signals from various sources, including the RF device 120, photoacoustic device 130, ECG 140 (if gating is enabled), and/or other devices. The analytic engine 160 may preprocess these signals to remove noise and artifacts that may interfere with the subsequent analysis, which may involve filtering, amplification, and other signal conditioning techniques.

The analytic engine 160 may analyze tissue displacement within the muscle compartment by tracking the movement of various tissue landmarks, such as the arterial wall and the fascia, in response to the pulsatile blood flow and/or the ARFI push. The analytic engine 160 may calculate the displacement amplitude, waveform, and frequency content of these movements, providing valuable information about tissue health and pressure within the compartment. In some embodiments, the analytic engine 160 may use ARFI 124 signals to measure tissue stiffness in the assessment of risk of compartment syndrome. In one example, by analyzing the tissue's response to the ARFI push, the analytic engine 160 may calculate the Young's modulus, a measure of tissue elasticity. Increased tissue stiffness, reflected in a higher Young's modulus, can be indicative of elevated compartment pressure and potential compartment syndrome.

In some embodiments, the analytic engine 160 may incorporate Doppler and photoacoustic data to assess blood flow and oxygenation within the compartment. Doppler signals may provide information about blood flow velocity and direction, while photoacoustic signals may measure blood oxygen saturation. These parameters may assist with evaluating tissue perfusion and identifying potential compromise due to elevated compartment pressure. In additional embodiments where ECG gating is enabled, the analytic engine 160 may synchronize the acquired data with the cardiac cycle by aligning the tissue displacement and other measurements with specific points in the heartbeat, such as systole and diastole phases. This temporal alignment reduces variability and improves the accuracy of the analysis, particularly in the assessment of tissue dynamics and blood flow.

The analytic engine's ability to integrate data from multiple modalities advantageously permits combining information from ultrasound, ARFI 124, Doppler, photoacoustics, ECG 140, and/or other sources to the analytic engine 160 to determine a comprehensive picture of tissue health within the compartment. This multi-modal approach advantageously enhances the accuracy and sensitivity of the diagnosis, enabling earlier detection and more effective monitoring of compartment syndrome without requiring physically intrusive procedures.

Based on the processed digital signal data, the analytic engine 160 may diagnostic information, which may indicate a risk profile for compartment syndrome. This may involve analyzing the displacement amplitudes in the artery tissue, compartment tissue, and fascia tissue, as well as other relevant parameters. The analytic engine 160 may categorize the risk into low, medium, or high, providing valuable information for guiding treatment decisions and monitoring the progression of the condition.

The analytic engine 160 may communicate the diagnostic information to a user terminal 170 for display to medical professionals to provide a summary of the diagnostic information and any determined displacement relationship. This may include numerical values, waveforms, images, indicators, risk profiles, trend analyses, and/or other displays that would be apparent to a person of skill in the art after having the benefit of this disclosure.

In various embodiments, additional processing of the displacement data may be used to diagnose and/or potentially stage compartment syndrome. The displacement resistance of the compartment may alter the waveform of the displacement signal to detectable levels. In some embodiments, shorter duration pulses may increase bandwidth of the displacement signal by increasing harmonic amplitudes and may be indicative of higher pressure within the compartment. In the same or other embodiments, monitoring the ratio of the amplitude of frequency components over multiple heartbeats may improve the staging and diagnostic value.

In some embodiments, the propagation of the displacement of the compartment can yield wave speeds which may directly measure stiffness of the tissue in the compartment. The ratio of the displacement amplitude through the compartment as well as the derivatives of spatial displacements through the compartment may show the stiffness of the tissue. In various embodiments, the displacement signals drifting over time may indicate that the compartment is growing, which may lead a user to conclude that the compartment syndrome is worsening.

In some embodiments, monitoring the displacement signals over extended periods of time may show the stages of compartment syndrome to an outside user. For example, as the tissue in the compartment continues to expand, it may be seen in an imaging plane. Comparing the total accumulated displacements across the same compartment can indicate earlier stages of compartment abnormalities. Later stages may show a higher pressure in the compartment and/or amplitude displacements in the compartment and/or on its surfaces, without limitation.

Various ARFI 124 measurements may also provide further indications for compartment syndrome. Shear wave velocity may directly measure the Youngs Modulus, without limitation. Other measurements, such as the total displacement, which may relate to relative stiffness, or the time the tissue takes to move some percentage of the total displacement, such as indicating viscoelasticity, may also be indicative of the relative pressure inside of the compartment, without limitation. In some embodiments, the ARFI push pulse may be substituted by or used in conjunction with observing movement of the artery.

In some embodiments, a pulse wave (PW) doppler technique covering a transverse slice of vasculature may yield volume flow analysis of the blood into and out of a compartment. For example, a system enabled by this disclosure may detect if blood moves despite total volume flow is low or dropping, which may be used to suggest a restriction is present. In some embodiments, integrating the power in the frequency bins over time may yield a net volume flow, particularly while the transducer 122 may be held at an intentional elevational angle, without limitation. For example, the process of this and other embodiments may be used with a plane wave imaging sequence, without limitation.

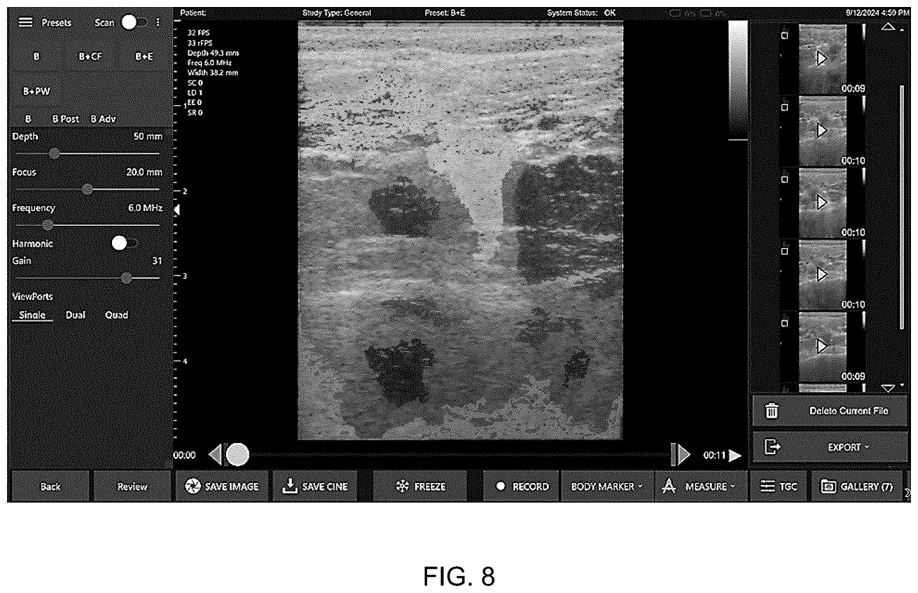

The user terminal 170 will now be discussed in greater detail. FIGS. 1 and 8 highlight examples of the user terminal 170, which may also be shown in other figures. The user terminal 170 may present at least part of the diagnostic information derived by the analytic engine 160 to an operator to indicate a likelihood of development of the adverse medical condition. The user terminal 170 may include a tilt sensor 172 or other aid to assist with orienting the transducer 122 to optimize efficacy by which the radio signals apply the induced displacements to the tissue and detect the responsive displacements of the tissue.

In at least one embodiment, the user terminal 170 may be provided to view and/or interact with diagnostic information determined by the analytic engine 160. In some embodiments, the user interface may be provided in a simple format, as displayed in FIG. 8. The screen of the user terminal 170 may include a touch screen. In some embodiments, a user may be able to move a first on-screen indicator, for example a virtual box, to encompass targeted tissue such as an artery. The user may additionally move a second on-screen indicator, for example an additional virtual box, to encompass a portion of the fascia. In other embodiments, the system may be automated, which may advantageously remove operator dependence and/or reduce the reliance on an operator to operate a system enabled by this disclosure.

A tilt indicator may be provided to reduce error by the operator when the transducer 122 is not tilted to intersect the artery at an optimal angle, which may approach 90 degrees, without limitation. A threshold value, for example maximum value, in the artery may be shaded and its current value may be indicated by a virtual needle. A user may adjust the tilt to a desired response, for example maximum response. An error reduction feature may be provided to supplement or replace the tilt indicator with data produced using an M-Mode screen. In some embodiments, an at least partially automated mechanical setup may be provided to assist with determining a peak elevational tilt. Optionally, a rock (heel and toe) indicator may be used to obtain a desired signal, for example maximum signal, from the fascia. This may be automated through multi-line acquisition methods or a “gate angle” adjusted by the user similar to PW doppler for compensation. In other embodiments, a physical mount or holding device may be used to position the transducer to maintain a desired relationship with the limb or other subject being observed.

In some embodiments, a user may discover that finding the peak elevational displacement angle is preferred, particularly for direct tissue, fascia, and/or arterial wall displacement measurements, without limitation. In the same or other embodiments, other measurements, such as the total volume flow in the vasculature, may be advantageous for an intentional elevational angle finding, without limitation. Guiding a user or automating the collection may advantageously improve usability and quality of results associated with operation of a system enabled by this disclosure.

The value for the fascia displacement, arterial displacement, compartment displacement, and/or other tissue displacements may be displayed to the user. Ins some embodiments, each identified target tissue may be displayed with an associated color to promote quick differentiation between tissue displacements. Alternatively, the value for the fascia displacement may be taken and/or displayed separately from the value of the arterial displacement, which may sequentially be determined using varied angles.

The user terminal 170 may provide a cine mode. In one example of a cine mode feature, the last ten seconds of image data may be stored in a memory and may be used in reviewing and determining the highest values. In some embodiments, a compartment syndrome detection mode may be provided as an integration of dual M-mode with RF data capture, displacement measurements, and filtering.

The photoacoustic device 130 will now be discussed in greater detail. FIG. 1 highlight examples of the photoacoustic device 130, which may also be shown in other figures. As will be appreciated by those of skill in the art, examples of photoacoustic devices 130 that may be used include light emitting devices, for example, light emitter LEDs, lasers, laser diodes, microwave emitters, EM waves associated with a radar or similar setup, and/or other photoacoustic devices 130, without limitation.

The compartment syndrome detection system may integrate a photoacoustic device 130 to enhance its diagnostic capabilities by providing information about blood oxygenation within the muscle compartment, supplementing the radio signals obtained from the RF device 120. The photoacoustic device 130 may operate by emitting a light pulse into the tissue to be absorbed by hemoglobin, a protein in red blood cells that carries oxygen. This absorption may generate a localized heating effect, leading to the production of acoustic waves that are detectable by an ultrasound transducer 122, providing information about the blood oxygenation levels within the tissue.

The integration of photoacoustics with the existing ultrasound and Doppler measurements may provide a multi-modal imaging system to improve an assessment of the physiological state of the tissues within the compartment. The analytic engine 160 may interpret the blood oxygenation data in conjunction with the radio signals and/or other signal modes to provide a more accurate diagnosis of compartment syndrome and/or other adverse medical conditions.

The photoacoustic device 130 may be communicably connected with the transducer 122, enabling synchronous detection of blood oxygenation data and radio signals from both modalities, which may be temporally correlated. The addition of blood oxygenation data to the diagnostic information enhances the system's ability to detect and stage compartment syndrome. For example, in compartment syndrome, increased pressure within the compartment can compromise blood flow, leading to a decrease in tissue oxygenation. By monitoring blood oxygenation levels, the system can provide an early indication of compromised blood flow, even before significant changes in tissue displacement or stiffness are detected. Furthermore, inclusion of a photoacoustic device 130 can be used to assess the severity of compartment syndrome by correlating tissue oxygenation level decreases with a risk profile. By monitoring the trend in blood oxygenation, the system can provide valuable information about the progression of the condition and the effectiveness of any interventions.

In various embodiments, photoacoustics may be determined using a light emitting device that operates using about 710-820 nm wavelength signals to detect the level of oxygenation in blood associated with a compartment. In other embodiments, photoacoustics may be used in wavelength signals that measure below 710 nm and/or above 820 nm. As will be appreciated by those of skill in the art, measurements relating to total hemoglobin, oxygenated blood, and/or deoxygenated blood can be used with various wavelength signals. For example, shorter wavelengths may be used despite having higher attenuation, for example using wavelengths below the 400 nm range. In another example, shallow compartments can be interrogated using longer wavelengths, such as those above an about 1500 nm range. Diode arrays with desired wavelength ranges may be fabricated and implemented into a system enabled by this disclosure to assist with determining a level of oxygenation, without limitation.

In one embodiment, LEDs (Light Emitting Diodes) or laser diodes can be used to construct an economical, compact patch device capable of averaging raw radio signals at very high pulse repetition rates, thereby reducing undesired noise. In one example where a distance of about 4 cm is used, with unidirectional sound travel, a theoretical maximum frequency of nearly 40 kHz may be achieved. The use of LEDs or laser diodes may advantageously provide low total power consumption, improving the safety of operation compared to conventional lasers, additionally allowing for effective noise reduction while enhancing signal strength at these frequencies. Moreover, motion may be extracted by also using shorter running average windows to “motion correct” data relating to longer time frames for oxygenation assessments. Variation in running average windows may additionally assist with deriving displacement signals for the analysis of amplitude and frequency content.

Photoacoustic analysis may advantageously provide useful information in deep tissue applications. For example, an end device could include two LEDs and a single transducer 122 element. In this example, range gating the received signal could provide information regarding oxygen (O2) content at a given depth that is otherwise unavailable using merely round-trip optics.

The multi-modal input signals will now be discussed in greater detail. The analytic engine 160 may derive the diagnostic data using multi-modal input signals 110 comprising at least two selected of ultrasound signals, acoustic radiation force impulse (ARFI 124) signals, photoacoustics, and/or ECG gating signals. In a multi-modal configuration, the analytic engine 160 may process input signals from various sources to derive comprehensive diagnostic data. For example, ultrasound signals may provide information about tissue structure and movement, including the motion of the artery and fascia walls. ARFI 124 signals may measure tissue stiffness, indicating the mechanical properties of the tissues within the compartment. Photoacoustic signals provide data on blood oxygenation levels, reflecting the physiological state of the tissues. ECG gating signals may synchronize the measurements with the cardiac cycle, reducing variability and improving accuracy. By combining these different modalities, the analytic engine 160 may create a more comprehensive picture of the condition within the compartment, allowing for a more accurate risk assessment of compartment syndrome.

In some embodiments, the radio signals may be gated with an electrocardiogram (ECG 140) to substantially correlate the induced displacement and responsive displacement with electrical cardiac activity respective to a point in a cardiac cycle. The analytic engine 160 may temporarily align and interpret a relationship between the point in the cardiac cycle and the digital signal data while deriving the diagnostic data. The analytic engine 160 may additionally interpret the responsive displacements of the tissue during systole and diastole of the cardiac cycle as indicated by the ECG 140. In some embodiments, the radio signals may be emitted and received by the RF device 120 using non-uniform sampling, which may be substantially correlated with the point in the cardiac cycle via gating with the ECG 140.

The use of ECG gating offers several advantages in the context of compartment syndrome risk assessment and/or detection. ECG gating may reduce variability in tissue displacement measurements caused by the pulsatile nature of blood flow by ensuring that the data is captured at determinable points in the heart's rhythm, leading to more reliable results. Additionally, ECG gating may improve the accuracy of tissue displacement measurements by isolating tissue response to the induced displacement from the natural motion caused by the heart's pumping action. Furthermore, ECG gating can help reduce noise in the radio signals by acquiring data only at designated points in the cardiac cycle and filtering out noise caused by other movements in the body, such as respiration or muscle contractions.

In addition to ECG gating, the compartment syndrome detection system may employ non-uniform sampling to further enhance its capabilities, which may acquire data at irregular intervals, rather than at a fixed rate. This non-uniform sampling technique can be advantageous in capturing transient events or rapid changes in the signal and/or diversifying the temporal information provided by various samples, which may be missed with uniform sampling.

The combination of ECG gating and non-uniform sampling may allow the system to capture a more detailed and accurate picture of tissue behavior within the compartment. By synchronizing the measurements with the cardiac cycle and acquiring data at irregular intervals, the system can effectively capture both the subtle and rapid changes in tissue displacement, providing valuable information for the diagnosis and monitoring of compartment syndrome.

The imaging stabilization components will now be discussed in greater detail. Registration tracing may be applied to identify a landmark of the radio signals and at least partially align the radio signals over time by matching the landmark to substantially stabilize the digital data signal used by the analytic engine 160 to derive the diagnostic information. Registration tracing may involve identifying a landmark in the radio signals and using it to align the signals over time, which may help stabilize the digital data signal used by the analytic engine 160 to derive diagnostic information. The landmark used for registration tracing can be virtually any distinct feature in the radio signals that can be reliably identified across multiple measurements, for example, an anatomical structure such as the arterial wall or the fascia, or it could be a specific pattern in the signal itself.

Once the landmark is identified, the system may apply registration tracing to align the radio signals over time. This involves applying transformations, such as translation, rotation, and scaling, to one signal to align it with another to substantially match the landmark in each signal, ensuring that the data is aligned and consistent across multiple measurements. The stabilized digital data signal may then be used by the analytic engine 160 to derive diagnostic information.

To further enhance the stability and accuracy of the compartment syndrome detection system, various transducer holders can be employed to minimize unwanted movement during imaging. These holders provide a secure and stable platform for the transducer 122, ensuring consistent positioning and reducing motion artifacts in the acquired data. One example of a transducer holder utilizes a tripod to provide a stable base for the device. The tripod's adjustable legs allow for flexible positioning and height adjustment, accommodating various patient anatomies and imaging locations. The transducer 122 may be securely mounted on the tripod head, which can be tilted and rotated to achieve the optimal imaging angle. Another type of holder may be designed to be mounted directly on the patient's limb.

In operation, a system enabled by this disclosure may be operated detect conditions in a patient indicative of compartment syndrome without the requirement of pulsed phase-locked loop devices or invasive procedures. Those of skill in the art will appreciate that the following methods are provided to illustrate an embodiment of the disclosure and should not be viewed as limiting the disclosure to only those methods or aspects. Skilled artisans will appreciate additional methods within the scope and spirit of the disclosure for performing the operations provided by the examples below after having the benefit of this disclosure.

A compartment syndrome detection system enabled by this disclosure may be operated to assist medical professionals in the diagnosis and monitoring of compartment syndrome. This example provides an illustrative walkthrough of how to operate a system enabled by this disclosure according to an illustrative embodiment and is provided without limitation. In this example, a transducer of the RF device may be located at a position on a patient where the radio signal will be used, for example, on a limb to be analyzed. A radio signal may be emitted from the RF device into the tissue of the subject, the reflection of which may be received as a radio signal. In some embodiments, the radio signal may be associated with ultrasound, Doppler, photoacoustic, and/or ECG signals if gating is enabled. The acquired radio signals may be translated from an analog electrical signal to digital signal data, which may be performed by a signal conversion device.

The analytic engine may process the acquired data, extracting relevant parameters such as tissue displacement, stiffness, blood flow, and oxygenation to determine diagnostic information. The results may be displayed on the user terminal, which may be accompanied by reference values and/or a trend analysis. The operator can use this information to assess the condition of the muscle compartment and make a diagnosis of compartment syndrome.

The illustrative system may provide a comprehensive assessment of tissue health within a muscle compartment, enabling the determination of risk profiles for compartment syndrome. In this example, the assessment involves analyzing the displacement amplitudes in three key tissue types: artery tissue, compartment tissue, and fascia tissue. Based on these measurements, the system may categorize the risk into three levels: low, medium, and high.

A low risk profile may be indicated by high displacement amplitude in the artery tissue, coupled with low displacement amplitudes in both the compartment tissue and fascia tissue. This pattern suggests that the artery is pulsating strongly, but the surrounding tissues maintain their elasticity and are not exhibiting significantly anomalous movement. This typically indicates a healthy compartment with normal tissue pressures.

A medium risk profile is characterized by high displacement amplitudes in all three tissue types: artery tissue, compartment tissue, and fascia tissue. This pattern suggests that the increased pressure within the compartment is causing all tissues to move in tandem with the pulsating artery and exhibit reduced rebound and elasticity. This typically indicates an early stage of compartment syndrome, where tissue pressures are elevated but not yet critical.

A high risk profile is identified by low displacement amplitudes in all three tissue types: artery tissue, compartment tissue, and fascia tissue. This pattern suggests that the pressure within the compartment is so high that it restricts the movement of all tissues, including the artery. This typically indicates a critical stage of compartment syndrome, where tissue pressures are severely elevated and blood flow is compromised.

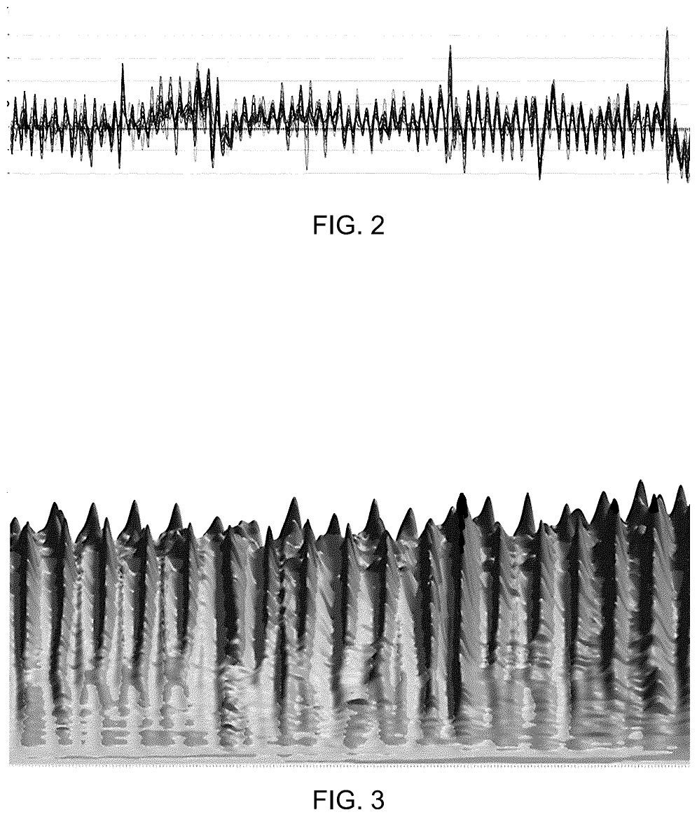

Referring to FIGS. 2-3, these graphs visualize two-dimensional (FIG. 2) and three-dimensional (FIG. 3) representations of an illustrative tissue displacement within a muscle compartment caused by the pulsating artery. The vertical axis represents depth, measured from the skin surface down to the back of the artery. Along this axis, distinct tissue layers can be identified: the superficial fat layer, the deeper muscle tissue, and the artery itself. The horizontal axis, on the other hand, represents the magnitude of tissue displacement resulting from the artery's pulsatile activity.

Examining the graph, it is observed that the fat layer, being more compressible, exhibits greater displacement compared to the denser muscle tissue. This difference in displacement reflects the varying stiffness of the tissues, with fat being more deformable and muscle being more resistant to deformation. Moving closer to the artery, the tissue displacement becomes more pronounced due to the direct pulsatile force exerted by the artery.

The upper portion of the graph displays the waveform of tissue displacement over time, capturing the rhythmic motion induced by the cardiac cycle. Each upward peak corresponds to the expansion phase of the artery, pushing the surrounding tissue outward. Conversely, the downward slopes represent the contraction phase of the artery, allowing the tissue to return to its resting position. This waveform analysis provides valuable information about the timing and characteristics of tissue movement, aiding in the assessment of compartment health.

To enhance the clarity and accuracy of the analysis, non-uniform sampling can be employed. This technique involves acquiring data at irregular intervals, specifically targeting points within the cardiac cycle that are most relevant to tissue motion. By averaging measurements from multiple heart cycles, the system minimizes the impact of noise and motion artifacts, resulting in a more precise representation of the tissue's true motion. Furthermore, this detailed analysis enables the detection of shear waves, which are mechanical waves that propagate through the tissue. By analyzing the characteristics of these shear waves, such as their velocity and attenuation, insights are gained into the tissue's stiffness and mechanical properties. This information complements the displacement data, providing a more comprehensive assessment of tissue health and the potential risk of compartment syndrome.

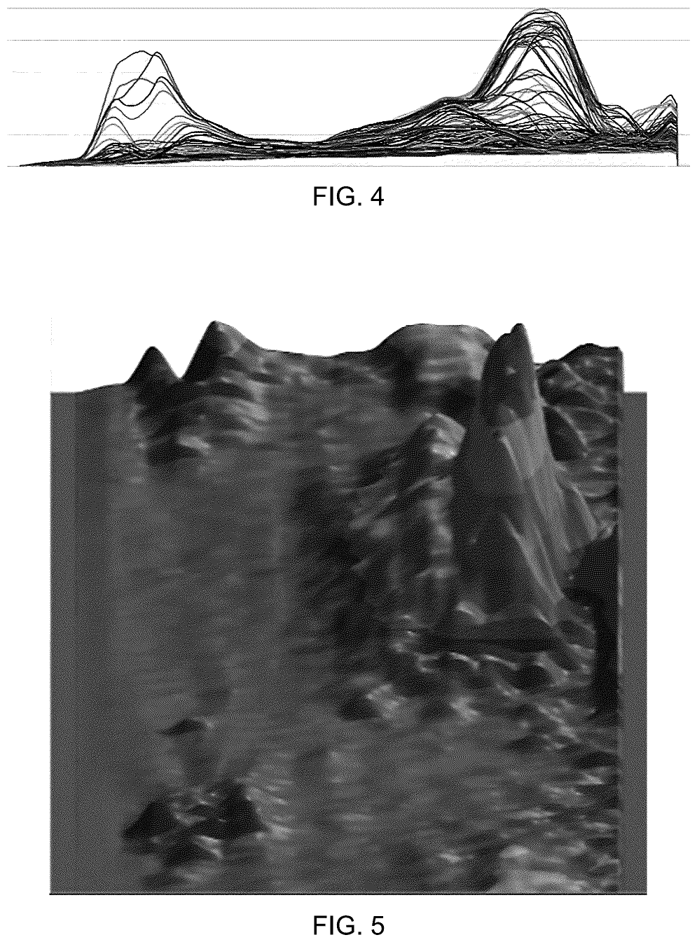

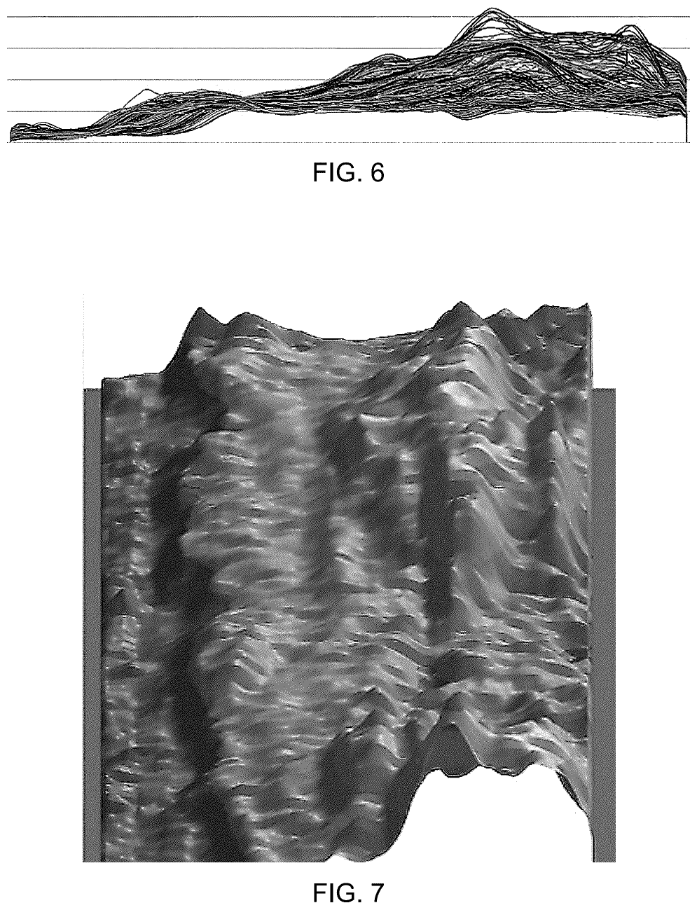

Referring to FIGS. 4-7, these graphs visualize two-dimensional (FIG. 4 for low risk and FIG. 6 for medium risk) and three-dimensional (FIG. 5 for low risk and FIG. 7 for medium risk) representations of the impact of pressure changes within a muscle compartment on tissue displacement and artery visibility. FIGS. 4 and 6 show individual acoustic lines, representing tissue depth from surface to the back of the artery. FIGS. 5 and 7 display an area representation of these lines from FIGS. 4 and 6, respectively, with depth on the vertical axis and lateral position on the horizontal axis.

In the “no pressure” graphs of FIGS. 4-5 associated with low risk, the artery is clearly visible with high amplitude pulsations that dissipate quickly into the surrounding tissue. This indicates that the tissue is soft and deforms easily in response to the artery's pulsation. Conversely, in the “medium pressure” graphs of FIGS. 6-7 associated with medium risk, the artery's pulsations are dampened, and the tissue displacement is more linear and less pronounced. This suggests that the increased pressure within the compartment is making the tissue stiffer and less deformable. The deformation is now concentrated at the boundary between the fascia and the fat layer, as the fat is more compressible than the hardened compartment.

These graphs of FIGS. 4-7 represent a composite average of multiple heartbeats, showing the total displacement over 10-20 cardiac cycles. This averaging helps to visualize the overall trend of tissue displacement and how it changes with increasing pressure. While these graphs effectively visualize tissue displacement, the ultimate goal is to extract meaningful diagnostic information. This can be achieved by color-coding the displacement data or using other visualization techniques to highlight areas of interest. Another important observation made evident by FIGS. 4-7 is the visibility of smaller arteries and vessels within the compartment. In the “no pressure” graphs of FIGS. 4-5, these vessels are more apparent, but they become dampened and less visible in the “medium pressure” graphs of FIGS. 6-7. This loss of visibility is another indicator of increased compartment pressure and potential compartment syndrome.

To improve the clarity of these smaller vessels, image registration techniques can be employed. By tracking the movement of these vessels over time and adjusting the averaging window accordingly, smearing effects caused by probe motion can be reduced and clearer visualization of their pulsations may be obtained. This enhanced visualization can help further aid in the diagnosis and monitoring of compartment syndrome.

Referring to FIG. 8, this diagram visualizes an illustrative interface from the user terminal that displays a visualization of tissue displacement and waveform analysis, providing insights into the severity of compartment syndrome. The visualization combines information from tissue displacement, deformation, and waveform analysis to highlight areas of concern. The circular area surrounding the artery represents the area of tissue displacement caused by the artery's pulsation. The size of this circular area, relative to the size of the artery, serves as an indicator of compartment pressure and tissue health. When compartment pressure is low, the circular area is small and tightly confined around the artery, indicating that the surrounding tissue is deforming minimally. However, as compartment pressure increases, the circular area expands, signifying that the displacement is spreading further from the artery. This expansion reflects the increasing stiffness of the tissue due to elevated pressure.

The ratio between the size of the circular area and the size of the artery is a significant metric for assessing the severity of compartment syndrome. A small ratio, where the circular area is only slightly larger than the artery, indicates a healthy compartment with low pressure. Conversely, a large ratio, where the circular area extends significantly beyond the artery, suggests elevated compartment pressure and potential compartment syndrome.

To generate this illustrative visualization, the system averaged displacement data over multiple heart cycles, typically around 60 frames, without limitation. However, for optimal results, averaging over a larger number of cycles is contemplated to capture a more comprehensive picture of tissue dynamics and reduce the impact of noise and motion artifacts. Stabilizing the transducer during image acquisition is helpful for accurate displacement tracking and waveform analysis. Clamping the transducer or employing image registration techniques can help minimize motion artifacts and improve the reliability of the measurements.

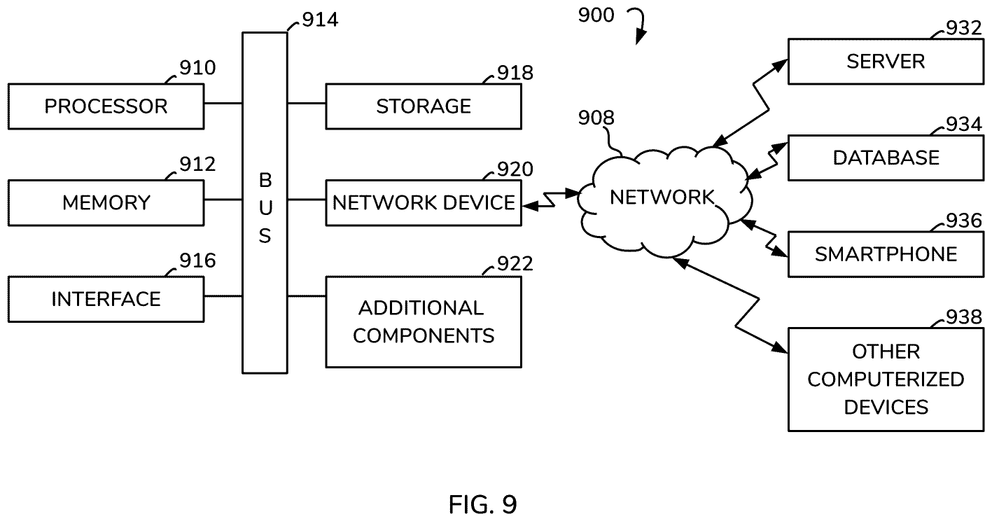

Referring now to FIG. 9, an illustrative computerized device will be discussed, without limitation. Various aspects and functions described in accord with the present disclosure may be implemented as hardware or software on one or more illustrative computerized devices 900 or other computerized devices. There are many examples of illustrative computerized devices 900 currently in use that may be suitable for implementing various aspects of the present disclosure. Some examples include, among others, network appliances, personal computers, workstations, mainframes, networked clients, servers, media servers, application servers, database servers and web servers. Other examples of illustrative computerized devices 900 may include mobile computing devices, cellular phones, smartphones, tablets, video game devices, personal digital assistants, network equipment, devices involved in commerce such as point of sale equipment and systems, such as handheld scanners, magnetic stripe readers, bar code scanners and their associated illustrative computerized device 900, among others. Additionally, aspects in accord with the present disclosure may be located on a single illustrative computerized device 900 or may be distributed among one or more illustrative computerized devices 900 connected to one or more communication networks.

For example, various aspects and functions may be distributed among one or more illustrative computerized devices 900 configured to provide a service to one or more client computers, or to perform an overall task as part of a distributed system. Additionally, aspects may be performed on a client-server or multi-tier system that include components distributed among one or more server systems that perform various functions. Thus, the disclosure is not limited to executing on any particular system or group of systems. Further, aspects may be implemented in software, hardware or firmware, or any combination thereof. Thus, aspects in accord with the present disclosure may be implemented within methods, acts, systems, system elements and components using a variety of hardware and software configurations, and the disclosure is not limited to any particular distributed architecture, network, or communication protocol.

FIG. 9 shows a block diagram of an illustrative computerized device 900, in which various aspects and functions in accord with the present disclosure may be practiced. The illustrative computerized device 900 may include one or more illustrative computerized devices 900. The illustrative computerized devices 900 included by the illustrative computerized device may be interconnected by, and may exchange data through, a communication network 908. Data may be communicated via the illustrative computerized device using a wireless and/or wired network connection.

Network 908 may include any communication network through which illustrative computerized devices 900 may exchange data. To exchange data via network 908, systems and/or components of the illustrative computerized device 900 and the network 908 may use various methods, protocols and standards including, among others, Ethernet, Wi-Fi, Bluetooth, TCP/IP, UDP, HTTP, FTP, SNMP, SMS, MMS, SS7, JSON, XML, REST, SOAP, RMI, DCOM, and/or Web Services, without limitation. To ensure data transfer is secure, the systems and/or modules of the illustrative computerized device 900 may transmit data via the network 908 using a variety of security measures including TSL, SSL, or VPN, among other security techniques. The illustrative computerized device 900 may include any number of illustrative computerized devices 900 and/or components, which may be networked using virtually any medium and communication protocol or combination of protocols.

Various aspects and functions in accord with the present disclosure may be implemented as specialized hardware or software executing in one or more illustrative computerized devices 900, including an illustrative computerized device 900 shown in FIG. 9. As depicted, the illustrative computerized device 900 may include a processor 910, memory 912, a bus 914 or other internal communication system, an input/output (I/O) interface 916, a storage system 918, and/or a network communication device 920. Additional devices 922 may be selectively connected to the computerized device via the bus 914. Processor 910, which may include one or more microprocessors or other types of controllers, can perform a series of instructions that result in manipulated data. Processor 910 may be a commercially available processor such as an ARM, x86, Intel Core, Intel Pentium, Motorola PowerPC, SGI MIPS, Sun UltraSPARC, or Hewlett-Packard PA-RISC processor, but may be any type of processor or controller as many other processors and controllers are available. As shown, processor 910 may be connected to other system elements, including memory 912, bus 914. The processor may additionally direct operation of a graphics processing unit (GPU), which may provide highly parallelized processing to increase the capability of analyzing large datasets.

The illustrative computerized device 900 may also include a network communication device 920. The network communication device 920 may receive data from other components of the computerized device to be communicated with servers 932, databases 934, smart phones 936, and/or other computerized devices 938 via a network 908. The communication of data may optionally be performed wirelessly. More specifically, without limitation, the network communication device 920 may communicate and relay information from one or more components of the illustrative computerized device 900, or other devices and/or components connected to the computerized device 900, to additional connected devices 932, 934, 936, and/or 938. Connected devices are intended to include, without limitation, data servers, additional computerized devices, mobile computing devices, smart phones, tablet computers, and other electronic devices that may communicate digitally with another device. In one example, the illustrative computerized device 900 may be used as a server to analyze and communicate data between connected devices.

The illustrative computerized device 900 may communicate with one or more connected devices via a communications network 908. The computerized device 900 may communicate over the network 908 by using its network communication device 920. More specifically, the network communication device 920 of the computerized device 900 may communicate with the network communication devices or network controllers of the connected devices. The network 908 may be, for example, the internet. As another example, the network 908 may be a WLAN. However, skilled artisans will appreciate additional networks to be included within the scope of this disclosure, such as intranets, local area networks, wide area networks, peer-to-peer networks, and various other network formats. Additionally, the illustrative computerized device 900 and/or connected devices 932, 934, 936, and/or 938 may communicate over the network 908 via a wired, wireless, or other connection, without limitation.

Memory 912 may be used for storing programs and/or data during operation of the illustrative computerized device 900. Thus, memory 912 may be a relatively high performance, volatile, random access memory such as a dynamic random access memory (DRAM) or static memory (SRAM). However, memory 912 may include any device for storing data, such as a disk drive or other non-volatile storage device. Various embodiments in accord with the present disclosure can organize memory 912 into particularized and, in some cases, unique structures to perform the aspects and functions of this disclosure.

Components of illustrative computerized device 900 may be coupled by an interconnection element such as bus 914. Bus 914 may include one or more physical busses (for example, busses between components that are integrated within a same machine), but may include any communication coupling between system elements including specialized or standard computing bus technologies such as USB, Thunderbolt, SATA, M.2, SCSI, PCI express, and other communication couplings. Thus, bus 914 may enable communications (for example, data and instructions) to be exchanged between system components of the illustrative computerized device 900.

The illustrative computerized device 900 also may include one or more interface devices 916 such as input devices, output devices and combination input/output devices. Interface devices 916 may receive input or provide output. More particularly, output devices may render information for external presentation. Input devices may accept information from external sources. Examples of interface devices include, among others, keyboards, ultrasound transducers, mouse devices, trackballs, magnetic strip readers, microphones, touchscreens, printing devices, display screens, speakers, network interface cards, etc. The interface devices 916 allow the illustrative computerized device 900 to exchange information and communicate with external entities, such as users and other systems.

Storage system 918 may include a computer readable and writeable nonvolatile storage medium in which instructions can be stored that define a program to be executed by the processor. Storage system 918 also may include information that is recorded, on or in, the medium, and this information may be processed by the program. More specifically, the information may be stored in one or more data structures specifically configured to conserve storage space or increase data exchange performance. The instructions may be persistently stored as encoded bits or signals, and the instructions may cause a processor to perform any of the functions described by the encoded bits or signals. The medium may, for example, be an optical disk, magnetic disk, or flash memory, among others. In operation, processor 910 or some other controller may cause data to be read from the nonvolatile recording medium into another memory, such as the memory 912, that allows for faster access to the information by the processor than does the storage medium included in the storage system 918. The memory may be located in storage system 918 or in memory 912. Processor 910 may manipulate the data within memory 912 and then copy the data to the medium associated with the storage system 918 after processing is completed. A variety of components may manage data movement between the medium and integrated circuit memory element and does not limit the disclosure. Further, the disclosure is not limited to a particular memory system or storage system.

Although the above described illustrative computerized device is shown by way of example as one type of illustrative computerized device upon which various aspects and functions in accord with the present disclosure may be practiced, aspects of the disclosure are not limited to being implemented on the illustrative computerized device 900 as shown in FIG. 9. Various aspects and functions in accord with the present disclosure may be practiced on one or more computers having components other than that shown in FIG. 9. For instance, the illustrative computerized device 900 may include specially-programmed, special-purpose hardware, such as for example, an application-specific integrated circuit (ASIC) tailored to perform a particular operation disclosed in this example. While another embodiment may perform essentially the same function using several general-purpose computing devices running Windows, Linux, Unix, Android, iOS, MAC OS, or other operating systems on the aforementioned processors and/or specialized computing devices running proprietary hardware and operating systems.

The illustrative computerized device 900 may include an operating system that manages at least a portion of the hardware elements included in illustrative computerized device 900. A processor or controller, such as processor 910, may execute an operating system which may be, among others, an operating system, one of the above mentioned operating systems, one of many Linux-based operating system distributions, a UNIX operating system, or another operating system that would be apparent to skilled artisans. Many other operating systems may be used, and embodiments are not limited to any particular operating system.

The processor and operating system may work together to define a computing platform for which application programs in high-level programming languages may be written. These component applications may be executable, intermediate (for example, C# or JAVA bytecode) or interpreted code which communicate over a communication network (for example, the Internet) using a communication protocol (for example, TCP/IP). Similarly, aspects in accord with the present disclosure may be implemented using an object-oriented programming language, such as JAVA, C, C++, C#, Python, PHP, Visual Basic .NET, JavaScript, Perl, Ruby, Delphi/Object Pascal, Visual Basic, Objective-C, Swift, MATLAB, PL/SQL, OpenEdge ABL, R, Fortran or other languages that would be apparent to skilled artisans. Other object-oriented programming languages may also be used. Alternatively, assembly, procedural, scripting, or logical programming languages may be used.

Additionally, various aspects and functions in accord with the present disclosure may be implemented in a non-programmed environment (for example, documents created in HTML5, HTML, XML, CSS, JavaScript, or other format that, when viewed in a window of a browser program, render aspects of a graphical-user interface or perform other functions). Further, various embodiments in accord with the present disclosure may be implemented as programmed or non-programmed elements, or any combination thereof. For example, a web page may be implemented using HTML while a data object called from within the web page may be written in C++. Thus, the disclosure is not limited to a specific programming language and any suitable programming language could also be used.

While various aspects have been described in the above disclosure, the description of this disclosure is intended to illustrate and not limit the scope of the invention. The invention is defined by the scope of the appended claims and not the illustrations and examples provided in the above disclosure. Skilled artisans will appreciate additional aspects of the invention, which may be realized in alternative embodiments, after having the benefit of the above disclosure. Other aspects, advantages, embodiments, and modifications are within the scope of the following claims.

What is claimed is:

1. A non-invasive adverse medical condition diagnostic system comprising:
a radio frequency (RF) device comprising an electro-acoustic transducer to emit and detect radio signals being at least partially within an ultrasound frequency range to detect responsive displacements in tissue resulting from induced displacements over a sampling duration; and
an analytic engine to interpret digital signal data indicative of the radio signals to derive diagnostic information from at least a displacement relationship between the responsive displacements of the tissue reacting to the induced displacements throughout at least part of the sampling duration; and
wherein a risk of an adverse medical condition is indicated via interpretation of at least the displacement relationship.

2. The system of claim 1:
wherein the tissue comprises:
artery tissue,
compartment tissue, and
fascia tissue;
wherein the adverse medical condition comprises compartment syndrome; and
wherein the risk is categorized comprising:
a low risk indicated by detecting high displacement amplitude in the artery tissue, low displacement amplitude in the compartment tissue, and low displacement amplitude in the fascia tissue,
a medium risk indicated by detecting high displacement amplitude in the artery tissue, high displacement amplitude in the compartment tissue, and high displacement amplitude in the fascia tissue, and
a high risk indicated by detecting low displacement amplitude in the artery tissue, low displacement amplitude in the compartment tissue, and low displacement amplitude in the fascia tissue.

3. The system of claim 1, further comprising:
a user terminal to present at least part of the diagnostic information derived by the analytic engine to an operator to indicate a likelihood of development of the adverse medical condition.

4. The system of claim 3, wherein the user terminal comprises:
a tilt sensor to assist with orienting the electro-acoustic transducer to optimize efficacy by which the radio signals apply the induced displacements to the tissue and detect the responsive displacements of the tissue.

5. The system of claim 1, further comprising:
a signal conversion device to adapt the radio signals detected by the RF device from an analog electrical signal to the digital signal data, further comprising filters to reduce unwanted interference comprising signal noise from the radio signals that are detected by the RF device to enhance the efficacy by which the digital signal data is interpreted by the analytic engine.

6. The system of claim 5, wherein the filters use digital signal processing to at least partially filter the digital signal data.

7. The system of claim 1:
wherein the radio signals are gated with an electrocardiogram (ECG) to substantially correlate the induced displacement and responsive displacement with electrical cardiac activity respective to a point in a cardiac cycle, and
wherein the analytic engine temporarily aligns and interprets a relationship between the point in the cardiac cycle and the digital signal data while deriving the diagnostic data.

8. The system of claim 7, wherein the analytic engine interprets the responsive displacements of the tissue during systole and diastole phases of the cardiac cycle as indicated by the ECG.

9. The system of claim 7:
wherein the radio signals are emitted and received by the RF device using non-uniform sampling; and
wherein the radio signals are substantially correlated with the point in the cardiac cycle via gating with the ECG.

10. The system of claim 1, further comprising:
a photoacoustic device to detect blood oxygenation data to supplement the radio signals of the RF device; and
wherein the analytic engine interprets a physiological state of the tissue indicated by the blood oxygenation data for the diagnostic information.

11. The system of claim 10, wherein the electro-acoustic transducer further comprises a light emitting device communicably connected with the photoacoustic device to detect the blood oxygenation data synchronously with detection of the radio signals by the RF device.

12. The system of claim 1, wherein the analytic engine derives the diagnostic data using multi-modal input signals comprising at least two selected from the group consisting of: ultrasound signals, acoustic radiation force impulse (ARFI) signals, photoacoustics, and/or ECG gating signals.

13. The system of claim 1, wherein registration tracing is applied to identify a landmark of the radio signals and at least partially align the radio signals over time by matching the landmark to substantially stabilize the digital data signal used by the analytic engine to derive the diagnostic information.

14. The system of claim 1, wherein the radio signal is detected by the RF device using B-mode to perform full image sampling of the tissue included within a sampling area.

15. A non-invasive compartment syndrome diagnostic system comprising:
a radio frequency (RF) device comprising a transducer to emit and detect radio signals to apply induced displacements in tissue and detect responsive displacements of the tissue over a sampling duration;
a signal conversion device to adapt the radio signals detected by the RF device from an analog electrical signal to digital signal data, the signal conversion device comprising filters to reduce unwanted interference comprising signal noise from the radio signals that are detected by the RF device; and
an analytic engine to interpret the digital signal data to derive diagnostic information from at least a displacement relationship between the responsive displacements of the tissue reacting to the induced displacements throughout at least part of the sampling duration; and
wherein the tissue comprises:
artery tissue,
compartment tissue, and
fascia tissue; and
wherein a risk of compartment syndrome is indicated via interpretation of at least the displacement relationship categorized comprising:
a low risk indicated by detecting high displacement amplitude in the artery tissue, low displacement amplitude in the compartment tissue, and low displacement amplitude in the fascia tissue,
a medium risk indicated by detecting high displacement amplitude in the artery tissue, high displacement amplitude in the compartment tissue, and high displacement amplitude in the fascia tissue, and
a high risk indicated by detecting low displacement amplitude in the artery tissue, low displacement amplitude in the compartment tissue, and low displacement amplitude in the fascia tissue.

16. The system of claim 1, further comprising:
a user terminal to present at least part of the diagnostic information derived by the analytic engine to an operator to indicate a likelihood of development of compartment syndrome; and
wherein a tilt sensor is included by the user terminal to assist with orienting the transducer to optimize efficacy by which the radio signals apply the induced displacements to the tissue and detect the responsive displacements of the tissue.

17. The system of claim 15:
wherein the radio signals are gated with an electrocardiogram (ECG) to substantially correlate the induced displacement and responsive displacement with electrical cardiac activity respective to a point in a cardiac cycle;
wherein the analytic engine temporarily aligns and interprets a relationship between the point in the cardiac cycle and the digital signal data while deriving the diagnostic data;
wherein the radio signals are emitted and received by the RF device using non-uniform sampling; and
wherein the radio signals are substantially correlated with the point in the cardiac cycle via gating with the ECG.

18. The system of claim 15, wherein registration tracing is applied to identify a landmark of the radio signals and at least partially align the radio signals over time by matching the landmark and substantially stabilize the digital data signal used by the analytic engine to derive the diagnostic information.

19. A non-invasive adverse medical condition diagnostic system comprising:
a radio frequency (RF) device comprising an ultrasonic transducer to emit and detect radio signals being at least partially within an ultrasound frequency range to apply induced displacements in tissue and detect responsive displacements of the tissue over a sampling duration;
an analytic engine to interpret digital signal data indicative of the radio signals to derive diagnostic information from at least a displacement relationship between the responsive displacements of the tissue reacting to the induced displacements throughout at least part of the sampling duration;
wherein the radio signals are gated with an electrocardiogram (ECG) to substantially correlate the induced displacement and responsive displacement with electrical cardiac activity respective to a point in a cardiac cycle;
wherein the analytic engine temporarily aligns and interprets a relationship between the point in the cardiac cycle and the digital signal data while deriving the diagnostic data; and
wherein a risk of an adverse medical condition is indicated via interpretation of at least the displacement relationship.

20. The system of claim 19, wherein the radio signal is detected by the RF device using B-mode to perform full image sampling of the tissue included within a sampling area.
